(19) United States
(12) Patent Application Publication
(10) Pub. No.: US 2013/0060128 A1
(43) Pub. Date: Mar. 7, 2013

(54) METHOD FOR OPERATING A RADIATION THERAPY SYSTEM

(71) Applicant: Joseph STANCANELLO, Gif sur Yvette (FR)

(72) Inventor: Joseph STANCANELLO, Gif sur Yvette (FR)

(73) Assignee: SIEMENS AKTIENGESELLSCHAFT, Munich (DE)

(21) Appl. No.: 13/602,479

(22) Filed: Sep. 4, 2012

(30) Foreign Application Priority Data
Sep. 6, 2011 (DE) 102011082181.3

(51) Int. Cl. A61N 5/10; A61B 5/055

(52) U.S. Cl. 600/411

(57) ABSTRACT

A method is disclosed for operating a radiation therapy system. The radiation therapy system includes a magnetic resonance apparatus. In an embodiment of the method, an irradiation plan is received for a patient. While the patient is arranged in the radiation therapy system and a hyperpolarized contrast agent has been administered, magnetic resonance data of the patient is acquired. In the magnetic resonance data, molecular irradiation target areas are determined on the basis of the hyperpolarized contrast agent and the received irradiation plan is corrected as a function of the molecular irradiation target areas determined in the magnetic resonance data. A molecular irradiation target area relates to an area of the patient with a molecular property.

### PRIORITY STATEMENT

The present application hereby claims priority under 35 U.S.C. §119 to German patent application number DE 10 2011 082 181.3 filed Sep. 6, 2011, the entire contents of which are hereby incorporated herein by reference.

### FIELD

At least one embodiment of the present invention generally relates to a method for operating a radiation therapy system and/or to a radiation therapy system, in which the method is used.

### BACKGROUND

Radiation therapy, which is also known as radiotherapy (RT), is a therapeutic approach based on ionizing radiation for the treatment of cancer for instance. Radiation therapy can however also be used to treat other diseases. Attempts are made in radiation therapy to supply an adequate therapeutic radiation dose to a diseased tissue, while surrounding healthy tissue is spared. The therapeutic effect is based on a different effect of the ionizing radiation on healthy and diseased tissue. A boundary area, a so-called margin, is usually added to the target area in order to ensure that position-related differences and movements between a planning phase and an irradiation phase do not influence the treatment result. In order conversely not to influence the surrounding healthy tissue with a specific target area variable including the boundary areas and a dose to be applied, the use of boundary areas restricts the maximum dose which can be supplied to the target area.

So-called image-guided radiation therapy (IGRT) was therefore introduced over the last few years. Image-guided radiation therapy enables the target area and surrounding healthy tissue, which may comprise so-called organs at risk (OAR), to be visualized before the supply of radiation so that boundary areas can theoretically be reduced. For the imaging radiation therapy, all known imaging techniques and imaging modalities are essentially used, such as for instance projective x-ray radiation, tomography x-ray radiation, ultrasound or magnetic resonance. Tomography x-ray imaging is however currently the widest spread, such as is shown for instance in the publication “A survey of image-guided radiation therapy use in the United States” by Simpson DR et al., 15. August 2010; 116(16): 3953-60.

On the other hand, in some clinical applications, the contrast of soft tissue using x-ray imaging is not sufficient, for instance at the points of contact between the bladder, prostate and stomach. In cases of this type, a combination of a radiation therapy system with a magnetic resonance imaging system can be used, such as is described for instance in the publication “MRI/linac integration” by Lagendijk J J et al. in Radiother Oncol. January 2008; 86(1): 25-9. The use of an imaging apparatus in combination with a radiation therapy apparatus can consequently be extended such that a functional or molecular item of information relating to the target area can be added to the anatomical information at the time of the treatment. As a result, a so-called biologically guided radiation therapy (BGRT) can be realized, which is described for instance in “BGRT: biologically guided radiation therapy—the future is fast approaching” by Steward RD et al. in Med Phys. October 2007; 34(10): 3739-51. Since magnetic resonance imaging nevertheless has a lower sensitivity in respect of molecular imaging, positron emission tomography (PET) is usually used for molecular imaging of a biologically guided radiation therapy. Positron emission tomography is however relatively complex.

### SUMMARY

At least one embodiment of the present invention provides a simpler and more cost-effective biologically guided radiation therapy, which ensures precise irradiation of the target area and minimal influence on healthy tissue during the radiation therapy treatment.

According to at least one embodiment of the present invention, a method for operating a radiation therapy system, a radiation therapy system, a computer program product and an electronically readable data carrier are disclosed. The dependent claims define preferred and advantageous embodiments of the invention.

According to at least one embodiment of the present invention, a method for operating a radiation therapy system is proposed. The radiation therapy system includes a magnetic resonance system for acquiring magnetic resonance data. In the method, an irradiation plan for a patient is received by the radiation therapy system. The irradiation plan may have been determined for instance in advance with the aid of an x-ray computed tomography system, a magnetic resonance tomography system or a positron emission tomography system. In a next step, magnetic resonance data of the patient is acquired, while the patient is arranged in the radiation therapy system. A hyperpolarized contrast agent is administered to the patient prior to the acquisition of magnetic resonance data. The hyperpolarized contrast agent may be produced in the treatment room or in close proximity to the treatment room and administered to the patient while he/she is arranged in the radiation therapy system. The hyperpolarized contrast agent provides for a significant increase in the signal-to-noise ratio in the acquired magnetic resonance data.

### BRIEF DESCRIPTION OF THE DRAWINGS

The invention is described below with reference to the drawings with the aid of preferred embodiments.

FIG. 1 shows a flow chart with method steps according to an embodiment of the present invention,

FIG. 2 shows a radiation therapy system according to an embodiment of the present invention.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

Before discussing example embodiments in more detail, it is noted that some example embodiments are described as processes or methods depicted as flowcharts. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Methods discussed below, some of which are illustrated by the flow charts, may be implemented by hardware, software, firmware, middleware, microcode, hardware description languages, or any combination thereof. When implemented in software, firmware, middleware or microcode, the program code or code segments to perform the necessary tasks will be stored in a machine or computer readable medium such as a storage medium or non-transitory computer readable medium. A processor(s) will perform the necessary tasks.

Specific structural and functional details disclosed herein are merely representative for purposes of describing example embodiments of the present invention. This invention may, however, be embodied in many alternate forms and should not be construed as limited to only the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In the following description, illustrative embodiments may be described with reference to acts and symbolic representations of operations (e.g., in the form of flowcharts) that may be implemented as program modules or functional processes include routines, programs, objects, components, data structures, etc., that perform particular tasks or implement particular abstract data types and may be implemented using existing hardware at existing network elements. Such existing hardware may include one or more Central Processing Units (CPUs), digital signal processors (DSPs), application-specific-integrated-circuits, field programmable gate arrays (FPGAs) computers or the like.

Note also that the software implemented aspects of the example embodiments may be typically encoded on some form of program storage medium or implemented over some type of transmission medium. The program storage medium (e.g., non-transitory storage medium) may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The example embodiments not limited by these aspects of any given implementation.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

According to at least one embodiment of the present invention, a method for operating a radiation therapy system is proposed. The radiation therapy system includes a magnetic resonance system for acquiring magnetic resonance data. In the method, an irradiation plan for a patient is received by the radiation therapy system. The irradiation plan may have been determined for instance in advance with the aid of an x-ray computed tomography system, a magnetic resonance tomography system or a positron emission tomography system. In a next step, magnetic resonance data of the patient is acquired, while the patient is arranged in the radiation therapy system. A hyperpolarized contrast agent is administered to the patient prior to the acquisition of magnetic resonance data. The hyperpolarized contrast agent may be produced in the treatment room or in close proximity to the treatment room and administered to the patient while he/she is arranged in the radiation therapy system. The hyperpolarized contrast agent provides for a significant increase in the signal-to-noise ratio in the acquired magnetic resonance data.

On the basis of the hyperpolarized contrast agent, molecular irradiation target areas in the magnetic resonance data are determined. A molecular irradiation target area includes an area of the patient with a predetermined molecular property. The molecular irradiation target area can include for instance a sub area of a diseased organ, which comprises predetermined molecular properties. Molecular properties of this type are also referred to as functional properties or biological properties and may include for instance an increased substance in the tissue, such as for instance increased lactate content or increased oxygen content or an increased molecular or biological activity in a tissue area.

The received irradiation plan is corrected as a function of the molecular irradiation target areas determined in the magnetic resonance data. The correction of the irradiation target areas may include for instance a geometric correction or a dosimetric correction. The received irradiation plan may already include molecular irradiation target areas. In this case, a position of the molecular irradiation target area in the received irradiation plan may be different to the irradiation target area determined in the magnetic resonance data for instance, or the molecular properties in the molecular irradiation target area determined in the magnetic resonance data may be different to those in the received irradiation plan. The irradiation plan may then be corrected as a function of the molecular irradiation target areas determined in the magnetic resonance data.

The use of the hyperpolarized contrast agent enables the molecular irradiation target areas to be advantageously highlighted in the magnetic resonance data, as a result of which signals, which relate to interfering background, such as for instance large water masses, can be smoothed out. The use of the hyperpolarized contrast agent enables a molecular activity of a tumor to be identified for instance. This molecular information can be acquired while the patient is lying on the patient couch in the same position, in which he/she is irradiated. It is possible in this way to ensure that an irradiation area is correctly aligned in respect of the treatment beams. In the event of a misalignment, geometric readjustment can be implemented for instance by a collimator. An adjustment of a dose can also be implemented. An improved radiation therapy can therefore be implemented on the basis of a biologically guided radiation therapy, if a hyperpolarized contrast agent for the magnetic resonance imaging is used in a combined apparatus consisting of a magnetic resonance system and a radiation therapy system. It may herewith be possible to reduce boundary areas of the target area and to increase a dose for the area of higher molecular activity, which is shown by recording the hyperpolarized contrast agent. As a result, the radiation therapy can be implemented more reliably and effectively.

According to an embodiment, the method further includes activation of a radiation generation apparatus for generating a beam to treat the patient arranged in the radiation therapy system according to the corrected irradiation plan. Since the patient is already in the radiation therapy system upon acquisition of the magnetic resonance data and correction of the irradiation plan, the beam generation apparatus can be actuated precisely such that irradiation target areas are found with high accuracy, as a result of which healthy surrounding tissue and organs at risk can be protected against the irradiation.

According to a further embodiment, the received irradiation plan includes, in addition to the molecular irradiation target areas, also anatomical irradiation target areas and areas of organs at risk. The anatomical irradiation target areas are also determined in the acquired magnetic resonance data and the received irradiation plan is corrected as a function of the molecular irradiation target areas determined in the magnetic resonance data and the anatomical irradiation target areas determined in the magnetic resonance data. Since anatomical areas can also be determined on the basis of the acquired magnetic resonance data, these anatomical irradiation target areas, for instance specific organs, and areas of organs at risk which are not to be irradiated, can be accurately detected and localized. The irradiation plan can be corrected on the basis of this addition anatomical information by for instance a beam alignment or a beam dose being adjusted. As a result, the boundary areas or margins mentioned in the introduction, which are provided between the irradiation target area and areas or areas of organs at risk which are not to be irradiated, can be reduced. A more effective and reliable irradiation of the patient can be implemented as a result.

The hyperpolarized contrast agent may include for instance a 13C pyruvate. This contrast agent provides for a significant increase in the signal-to-noise ratio in the acquired magnetic resonance data for an irradiation target area for instance, such as e.g. a tumor area. Higher molecular activity of a tumor area may be indicated for instance by an increased absorption of the 13C pyruvate. The hyperpolarized contrast agent can be based on helium (3He) or xenon (129Xe) instead of carbon (13C).

According to a further embodiment, the radiation therapy system includes a linear accelerator, a so-called linear accelerator (LINAC), or a cobalt-60 source (Co-60).

According to an embodiment of the present invention, a radiation therapy system is also provided, which includes a beam generation apparatus for generating a beam or radiation for the treatment of a patient arranged in the radiation therapy system and a magnetic resonance apparatus for acquiring magnetic resonance data of the patient arranged in the radiation therapy system. The radiation therapy system also includes a processing apparatus, which is coupled to the beam generation apparatus and the magnetic resonance apparatus in order to control these apparatuses. The processing apparatus is able to receive an irradiation plan for the patient.

The irradiation plan may have been created for instance in a preexamination of the patient. The irradiation plan may have been created for instance by using an x-ray computed tomography system, a magnetic resonance tomography system or a positron emission tomography system. The irradiation plan may be input directly into the processing apparatus for instance or transmitted for instance from a so-called oncology information system (OIS) to the processing apparatus.

The irradiation plan includes molecular irradiation target areas. A molecular irradiation target area includes an area of the patient with a predetermined molecular property, for instance specific molecular or biological activity. The irradiation plan may furthermore also include anatomical irradiation target areas or areas not to be irradiated, in particular areas of organs at risk.

The processing apparatus is also able to activate the magnetic resonance apparatus in order to acquire magnetic resonance data of the patient. The magnetic resonance data is acquired while the patient is arranged in the radiation therapy system. Prior to acquisition of the magnetic resonance data, a hyperpolarized contrast agent is administered to the patient, which is therefore in the body of the patient during the acquisition of magnetic resonance data, and was absorbed particularly well for instance particularly in the areas of high molecular activity, for instance a tumor area.

The molecular irradiation target areas are determined in the magnetic resonance data on the basis of the hyperpolarized contrast agent. In particular, a precise position of the molecular irradiation target areas can be accurately determined by a high signal-to-noise ratio on account of the hyperpolarized contrast agent in the magnetic resonance data. The irradiation plan is corrected as a function of the molecular irradiation target areas determined in the magnetic resonance data.

The radiation therapy system described previously is also suited to implementing the method described previously and therefore includes the advantages described in conjunction with the method.

An embodiment of the present invention further provides for a computer program product, in particular a computer program or software, which can be loaded into memory of a programmable processing apparatus of the radiation therapy system. The processing apparatus may include for instance a microprocessor or a computer. All or various of the previously described embodiments of the inventive method can be executed with this computer program product, if the computer program product is executed in the processing apparatus. In this way the computer program product possibly requires program means, for instance libraries or auxiliary functions, in order to realize corresponding embodiments of the method. In other words, a computer program or software should be protected with the claim focused on the computer program product, with which one of the afore-described embodiments of the inventive method can be executed and/or which executes the embodiment. In this process the software may be a source code, for instance C++, which has to be compiled or translated or bound again or which has only to be interpreted, or is an executable software code, which is only to be loaded into the corresponding processing apparatus for execution.

An embodiment of the present invention finally provides an electronically readable data carrier, for instance a CD, a DVD, a magnetic band or a USB stick, on which electronically readable control information, in particular software, as was described previously, is stored. If this control information and/or the software is read from the data carrier and stored in the processing apparatus, all inventive embodiments of the described method can be implemented.

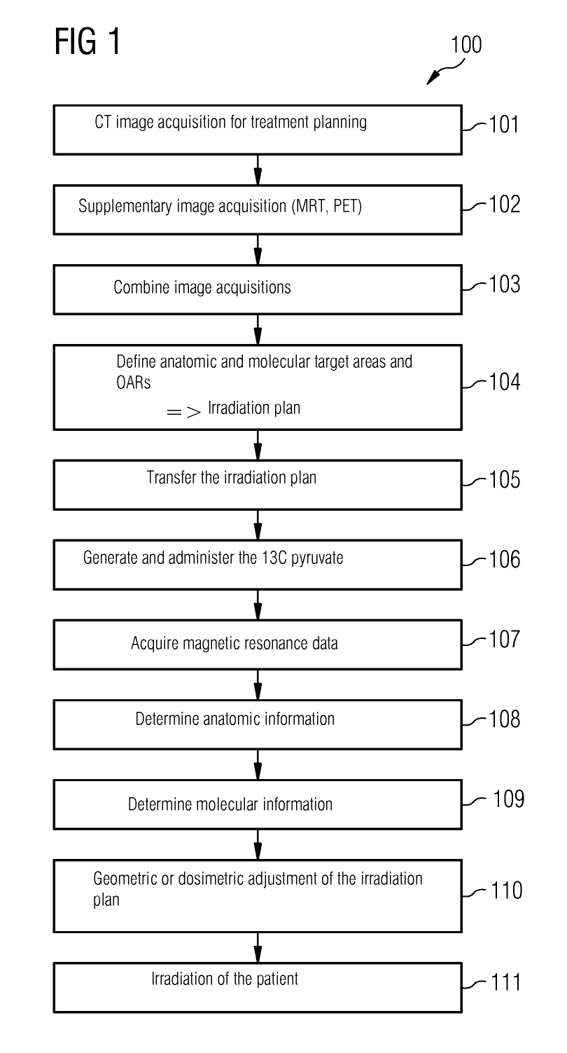

With reference to FIG. 1, an inventive method 100 is described, which uses molecular imaging abilities of a hyperpolarized contrast agent on the basis of a magnetic resonance imaging in combination with a radiation therapy apparatus. The molecular information enables an improved radiation therapy, which is guided by biological information, which is available during the radiation treatment. This enables safety boundary areas, which surround the target area, to reduce and/or increase a radiation dose, in order thus to enable a more effective and reliable radiation therapy. The method also enables the replacement of for instance apparatuses consisting of a combination of a positron emission tomography system and a radiation therapy apparatus. The thus enabled magnetic resonance-based molecular imaging can be used directly during a patient treatment time.

Treatment of a patient can be implemented as follows for instance. In step 101, a treatment plan or an irradiation plan based on an x-ray computed tomography image acquisition is created in a treatment planning phase. Additional image acquisitions, for instance with the aid of a magnetic resonance tomography system or a positron emission tomography system, can be implemented in step 102 and combined in step 103 with the x-ray computed tomography image acquisition system in step 101. The combination of the image acquisitions of the different image acquisition systems or image acquisition modalities is also referred to as “registration”. A treatment plan or irradiation plan is created on the basis of this information in step 104, which defines anatomical and molecular target areas as well as areas not to be irradiated or to be protected from radiation, for instance organs at risk. The irradiation plan or treatment plan can be stored for instance in a so-called oncology information system (OIS).

In step 105, the irradiation plan is transferred to a radiation therapy system. Alternatively, information relating to the irradiation plan can also be input directly into the radiation therapy system. The radiation therapy system includes a magnetic resonance system for acquiring magnetic resonance data and for determining magnetic resonance images on the basis of the acquired magnetic resonance data.

In step 106, a hyperpolarized contrast agent, for instance a 13C-pyruvate, is generated and administered to the patient. At this point in time, the patient may already be in the radiation therapy system or is positioned in the radiation therapy system following administration of the contrast agent.

In step 107 magnetic resonance data is acquired, while the patient is in the treatment position. On the basis of the acquired magnetic resonance data, current anatomical information of the patient is determined in step 108, as arranged in the radiation therapy system. Furthermore, current molecular information of the patient arranged in the radiation therapy system is also determined in step 109. The molecular information is determined by using the hyperpolarized contrast agent. A significantly increased signal-to-noise ratio may exist for instance in areas in which the hyperpolarized contrast agent has accumulated. The signal-to-noise ratio may be increased by the factor 10000 for instance. Since for instance areas in particular with a high molecular activity, for instance tumor areas, have an increased absorption of the hyperpolarized contrast agent, these areas can be very accurately determined in the acquired magnetic resonance data.

On the basis of the currently determined anatomical information and molecular information of the patient arranged in the radiation therapy system, the irradiation plan received in step 105 can be corrected in step 110. To this end, a geometric adjustment of the irradiation plan to the actual position of the areas to be irradiated and the areas not to be irradiated can be implemented for instance. Furthermore, an adjustment of the irradiation dose can be implemented based for instance on the molecular information.

In step 111, the treatment of the patient, i.e. the irradiation of the patient, is finally implemented according to the corrected irradiation plan.

A particularly reliable irradiation can then be implemented in particular if the patient is not repositioned between the acquisition of the magnetic resonance data in step 107 and the irradiation of the patient in step 111, i.e. if the irradiation of the patient is implemented immediately and at the same location after the acquisition of the magnetic resonance data and the correction of the irradiation plan. In order to ensure the hyperpolarized property of the contrast agent, manufacture of the hyperpolarized contrast agent in the immediate vicinity of the radiation therapy system is advantageous.

The previously described method can also be applied to other work flows, which are based on magnetic resonance imaging, for instance in a work flow in which the generation of the hyperpolarized contrast agent and the acquisition of the magnetic resonance data are implemented at a location which differs from the irradiation.

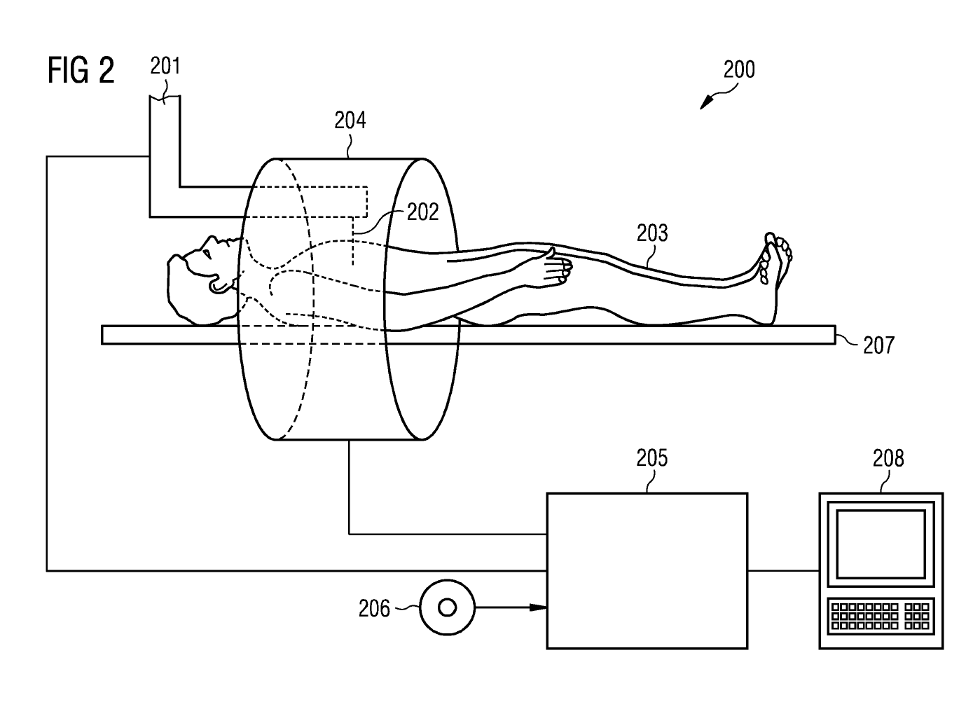

FIG. 2 shows a schematic representation of a radiation therapy system 200, which includes a beam generation apparatus 201 and a magnetic resonance apparatus 204. The radiation generation apparatus 201 is used to generate a particle beam 202 or an electromagnetic radiation 202 for the treatment of a patient 203 arranged in the radiation therapy system 200. The patient is mounted for instance on a moveable patient couch 207. The radiation generation apparatus 201 may include for instance a linear accelerator (LINAC), or a radiation source, for instance a cobalt-60 radiation source. The magnetic resonance apparatus 204 is used to acquire magnetic resonance data of the patient 203 arranged in the radiation therapy system 200. The acquisition of magnetic resonance data of the patient 203 and the generation of magnetic resonance images from the magnetic resonance data is known to a person skilled in the art and is therefore not described in detail herein. The radiation therapy system 200 further includes a processing apparatus 204, which is coupled to the magnetic resonance apparatus 205 and the radiation generation apparatus 201 in order to control the same. The processing apparatus 205 for instance includes a microprocessor or a programmable control apparatus, which can execute a program, for instance software. The program or the software can be loaded into the processing apparatus 205 with the aid of a data carrier 206 for instance. The processing apparatus 205 is also coupled to an oncology information system 208, in order for instance to receive the irradiation plan determined in step 104 from the oncology information system 208.

During operation, the processing apparatus 205 is able to implement the step of the method shown in FIG. 1. In particular, the processing apparatus 205 is able to receive an irradiation plan for the patient 203, which is positioned on a patient couch 207 in the radiation therapy system 200, from the oncology information system 208. The irradiation plan includes molecular irradiation target areas, which include areas of the patient 203 with a predetermined molecular property. While the patient is arranged in the radiation therapy system 200 and a hyperpolarized contrast agent has been administered, the processing apparatus 205 controls the magnetic resonance apparatus 204 in order to acquire magnetic resonance data of the patient 203. On the basis of the magnetic resonance data, the molecular irradiation target areas are determined for the current position of the patient 203. The hyperpolarized contrast agent enables this since the irradiation target areas, on account of their increased molecular or biological activity, absorb a particularly large quantity of the hyperpolarized contrast agent. The irradiation plan is corrected on the basis of the molecular irradiation target areas determined in the magnetic resonance data. An irradiation of the patient 203 then takes place with the corrected data of the irradiation plan.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for operating a radiation therapy system, the radiation therapy system including a magnetic resonance apparatus, the method comprising:
receiving an irradiation plan for a patient;
acquiring magnetic resonance data of the patient while the patient is arranged in the radiation therapy system, a hyperpolarized contrast agent being administered prior to acquiring the magnetic resonance data;
determining molecular irradiation target areas in the magnetic resonance data on the basis of the hyperpolarized contrast agent, wherein a molecular irradiation target area includes an area of the patient with a molecular property; and
correcting the received irradiation plan as a function of the molecular irradiation target areas determined in the magnetic resonance data.

2. The method of claim 1, wherein the irradiation plan for the patient includes molecular irradiation target areas, and wherein the correction of the received irradiation plan includes a correction of the molecular irradiation target areas of the irradiation plan as a function of the molecular irradiation target areas determined in the magnetic resonance data.

3. The method of claim 1, further comprising:
activating a radiation generation apparatus for generating a beam for the treatment of the patient arranged in the radiation therapy system according to the correction irradiation plan.

4. The method of claim 1, wherein the received irradiation plan includes anatomical irradiation target areas and areas of organs at risk, wherein the method further comprises:
determining the anatomical irradiation target areas in the magnetic resonance data, wherein the received irradiation plan is corrected as a function of the molecular irradiation target areas determined in the magnetic resonance data and the anatomical irradiation target areas determined in the magnetic resonance data.

5. The method of claim 1, wherein the hyperpolarized contrast agent includes a 13C pyruvate.

6. The method of claim 1, wherein the molecular property includes at least one of a lactate content, an oxygen content and molecular activity of a tissue area.

7. The method of claim 1, wherein the radiation therapy system includes a linear accelerator or a cobalt-60 source.

8. A radiation therapy system, comprising:
a radiation generation apparatus configured to generate a beam for the treatment of a patient arranged in the radiation therapy system;
a magnetic resonance apparatus configured to acquire magnetic resonance data of the patient arranged in the radiation therapy system; and
a processing apparatus, operatively coupled to the radiation generation apparatus and the magnetic resonance apparatus, wherein the processing apparatus is embodied to
receive an irradiation plan for the patient,
acquire magnetic resonance data of the patient, while the patient is arranged in the radiation therapy system, a hyperpolarized contrast agent being administered prior to the acquisition of the magnetic resonance data,
determine molecular irradiation target areas in the magnetic resonance data based on the hyperpolarized contrast agent, wherein a molecular irradiation target area includes an area of the patient with a molecular property, and
correct the irradiation plan as a function of the molecular irradiation target areas determined in the magnetic resonance data.

9. A computer readable medium including program segments for, when executed on a computer device, causing the computer device to implement the method of claim 1.

10. A computer program product, loadable directly into a memory of a processing apparatus of a radiation therapy system, including program segments in order to execute the method of claim 1 upon the program being executed in a processing apparatus of the radiation therapy system.

11. Electronically readable data carriers with electronically readable control information stored thereupon, embodied such that upon use of the data carrier in a processing apparatus of a radiation therapy system, the electronically readable data carriers execute the method of claim 1.

12. The method of claim 2, further comprising:
activating a radiation generation apparatus for generating a beam for the treatment of the patient arranged in the radiation therapy system according to the correction irradiation plan.

13. The method of claim 2, wherein the received irradiation plan includes anatomical irradiation target areas and areas of organs at risk, wherein the method further comprises:
determining the anatomical irradiation target areas in the magnetic resonance data, wherein the received irradiation plan is corrected as a function of the molecular irradiation target areas determined in the magnetic resonance data and the anatomical irradiation target areas determined in the magnetic resonance data.

14. The method of claim 2, wherein the hyperpolarized contrast agent includes a 13C pyruvate.

15. The method of claim 2, wherein the molecular property includes at least one of a lactate content, an oxygen content and molecular activity of a tissue area.

16. The method of claim 2, wherein the radiation therapy system includes a linear accelerator or a cobalt-60 source.
